(19) United States
(12) Patent Application Publication
(10) Pub. No.: US 2020/0129434 A1
(43) Pub. Date: Apr. 30, 2020

(54) LIPOSOME COMPOSITIONS AND USES THEREOF

(71) Applicant: TECHNION RESEARCH & DEVELOPMENT FOUNDATION LIMITED, Haifa (IL)

(72) Inventors: Yeshayahu KATZ, Haifa (IL); Moshe GAVISH, Tel Aviv (IL); Nahum ALLON, Macabim (IL); Martin GABAY, Haifa (IL); Meygal KAHANA, Tiberias (IL)

(21) Appl. No.: 16/605,601

(22) PCT Filed: Apr. 18, 2018
(86) PCT No.: PCT/IL2018/050444

§ 371 Date: Oct. 16, 2019

Related U.S. Application Data
(60) Provisional application No. 62/487,528, filed on Apr. 20, 2017.

(51) Int. Cl. A61K 9/127; A61K 31/12; A61K 31/495

(52) U.S. Cl. CPC A61K 9/1271; A61K 45/06; A61K 31/495; A61K 31/12

(57) ABSTRACT

The present invention provides compositions comprising liposomes comprises cholesterol, phosphatidyl phosphoric acid and phosphatidyl choline, as well as liposomes comprising a drug or imaging agent and a peptide for targeting to the brain. The invention further provides methods for treating or ameliorating a brain disease by administering the compositions of the invention.

### FIELD OF INVENTION

The present invention is directed to the field of liposome composition and drug delivery across the blood brain barrier.

### BACKGROUND OF THE INVENTION

Neurodegenerative diseases, cancer and infections of the brain become more prevalent as populations become older. However, despite the brain's relatively high blood flow, it is one of the least accessible organs for the delivery of active pharmacological compounds. There are two physiological barriers separating the brain from its blood supply and they control the entry and exit of endogenous and exogenous compounds. One is the blood-brain barrier (BBB) and the other is the blood-cerebrospinal fluid barrier (BCSFB). Since the surface area of the human BBB is estimated to be 5000 times greater than that of the BCSFB, the BBB is considered to be the main region controlling the uptake of drugs into the brain parenchyma and the target for delivering drugs to the brain.

The BBB is defined by the microvasculature of the brain, which consists of a monolayer of polarized endothelial cells connected by complex tight junctions. The function of the BBB is dynamically regulated by various cells, including astrocytes, neurons and pericytes. The endothelial cells are separated from these other cells by a basal lamina, whose components such as type IV collagen, laminin, fibronectin and heparan sulfate may be involved in drug transport, as some of them provide a negatively charged interface.

Nevertheless, methods of drug delivery across the BBB are still limited and not highly effective. Out of the 7000 drugs in the clinical pharmacopeia only about 5% penetrate the BBB. And those drugs that can be delivered have only been found to have mild efficacy in treated brain diseases. Novel drugs, and methods for delivery of these drugs directly to the brain are sorely needed.

### SUMMARY OF THE INVENTION

The present invention provides compositions comprising a liposome comprising cholesterol, phosphatidyl phosphoric acid and phosphatidyl choline, as well as a composition comprising a liposome further comprising a drug or imaging agent and a peptide for targeting to the brain. The invention also provides methods of treating or ameliorating a brain disease comprising administering to a subject a pharmaceutical composition comprising the compositions of the invention.

According to a first aspect, there is provided a composition comprising a liposome, the liposome comprises 30 to 50% cholesterol, 5 to 20% phosphatidyl phosphoric acid and 40 to 60% phosphatidyl choline, by molarity.

In some embodiments, the liposome's diameter is between 10 to 200 nm.

According to another aspect, there is provided a method of treating or ameliorating a brain disease in a subject in need thereof comprising: administering a pharmaceutical composition comprising any one of the compositions of the present invention and a pharmaceutically acceptable carrier or excipient to the subject, thereby treating the brain disease.

According to another aspect, there is provided a method for extending the half-life of a drug or imaging agent in a body of a subject, comprising: administering a pharmaceutical composition comprising any one of the compositions of the present invention and a pharmaceutically acceptable carrier or excipient to the subject, thereby extending the half-life of a drug or imaging agent in the body of a subject.

In some embodiments of the compositions and methods of the invention, the liposome further comprises a peptide anchored and conjugated to a succinate within the liposome bilayer, and the peptide is exposed to the outer surface of the liposome. In some embodiments, the peptide comprises the amino acid sequence HRERMS (SEQ ID NO: 1), the succinate is 1,2-dioleoyl-sn-glycero-3-succinate and the peptide anchored and conjugated to 1,2-dioleoyl-sn-glycero-3-succinate comprises 0.1-2% of the liposome, by molarity.

In some embodiments, the composition is for use in transport across the blood brain barrier (BBB).

In some embodiments of the compositions and methods of the invention, the liposome further comprises a drug or an imaging agent. In some embodiments, the liposome comprises 0.1 to 10% drug or imaging agent by molarity. In some embodiments, the drug or imaging agent is hydrophilic and encapsulated by said liposome. In some embodiments, the drug or imaging agent is hydrophobic and embedded in the lipid layer of said liposome.

In some embodiments of the compositions and methods of the invention, the drug is a central nervous system (CNS) drug or a brain therapeutic agent. In some embodiments, the CNS drug is selected from the group consisting of: a brain cancer therapeutic, a Parkinson's disease therapeutic, a Huntington's disease therapeutic, and an Alzheimer's disease therapeutic. In some embodiments, the brain cancer is glioblastoma.

In some embodiments of the compositions and methods of the invention, the drug is selected from the group consisting of: curcumin and temozolomide (TMZ). In some embodiments, the drug comprises TMZ. In some embodiments, the drug comprises curcumin. In some embodiments, the TMZ is present in a dose of 0.1 to 20 mg/m².

In some embodiments, the methods of the invention further comprise administering a cancer therapy selected from the group consisting of: radiation therapy, and chemotherapy.

### BRIEF DESCRIPTION OF THE DRAWINGS

FIG. 1. MRI images of mice, 8 days after cancer cell injection (left panel) and 22 days after cell injection (right panel). Arrows indicate the borders of the developing tumor.

FIG. 2. IVIS images of mice 18 days after the beginning of treatment. Free TMZ (left mouse), saline (middle mouse) and TMZ-liposomes (right mouse) were administered daily by intraperitoneal injection (4 mg/kg).

FIGS. 3A-3B. Line graphs depicting average radiance of the luciferine-luciferase reaction as measured by the IVIS 200 imaging system. Fluorescence was measured at various time points following the initiation of dosing. (3A) Tumor cell growth during dosing with TMZ-liposomes (red line), free TMZ (blue line) and saline (black line) was measured. (3B) Tumor cell growth during dosing with curcumin-liposomes (blue line), curcumin-liposomes with scrambled targeting peptide (red line), free TMZ (green line) and saline (black line) was measured.

FIG. 4. Survival plot showing the percent of surviving mice after transfer of U87 glioblastoma cells. Control—saline (black line), Free TMZ (red line), Liposomes containing TMZ (beige line), Liposomes containing Curcumin (blue line), Liposomes containing curcumin with scrambled target peptide (purple line), Free curcumin (pink line). 4 mg drug/kg mouse/treatment given daily.

FIG. 5. Line graph showing that the PA concentration is crucial for obtaining a stable negative zeta potential. As can be seen, the optimal concentration range of PA within a liposome was 12 to 18 mol %.

### DETAILED DESCRIPTION OF THE INVENTION

The present invention provides, in some embodiments, a liposome comprising cholesterol, phosphatidyl phosphoric acid, phosphatidyl choline and a peptide conjugated to 1,2-dioleoyl-sn-glycero-3-succinate, and pharmaceutical compositions and uses thereof.

### Liposome Composition

By one aspect, the present invention concerns a liposome, wherein cholesterol comprises 30-50% of the liposome by molar ratio, mol % or weight %, phosphatidyl phosphoric acid comprises 5-20% of the liposome by molar ratio, mol % or weight % and phosphatidyl choline comprises 40-60% of the liposome by molar ratio, mol % or weight %.

In one embodiment, a liposome as described herein comprises 30-50% cholesterol of the liposome by molar ratio, mol % or weight %.

The term “liposome” as used herein refers to an artificial small spherical vesicle comprised of lipid molecules enclosing a hydrophilic center. In some embodiments, the vesicles lack a hydrophilic center. In some embodiments, the vesicles are micelles. In some embodiments, the liposome comprises a lipid monolayer. In some embodiments, the liposome comprises a lipid bilayer.

The term “molarity” as used herein refers to the percentage of molecules of a substance relative to all the molecules that comprise the liposome. Further, the relative amounts are provided by the number of molecules (as given by the number of moles) of each substance. In some embodiments, the molarity can be provided as a percent of the total number of moles of all substances that make-up the liposome. Such a percentage can be abbreviated mol % or weight %. In one embodiment, molarity is mol %. In one embodiment, weight % comprises w/w %.

In some embodiments, the liposome comprises 40-50%, 30-50%, 20%-50%, 10-50%, 40-60%, 30-60%, 20-60%, 35-50%, 40-50%, 30-45%, or 30-40% cholesterol by molarity or weight. Each possibility represents a separate embodiment of the present invention. In some embodiments, 40-50%, 30-50%, 20%-50%, 10-50%, 40-60%, 30-60%, 20-60%, 35-50%, 40-50%, 30-45%, or 30-40% of all the molecule of the liposome are cholesterol. Each possibility represents a separate embodiment of the present invention. In some embodiments, the liposome comprises 10-45%, 15-45%, 20-45%, 25-45%, 30-45%, 10-40%, 15-40%, 20-40%, 25-40%, 30-40%, 10-35%, 15-35%, 20-35%, 25-35%, 30-35%, 10-30%, 15-30%, 20-30%, or 25-30% cholesterol, by weight or mol %. Each possibility represents a separate embodiment of the present invention. In some embodiments, the liposome comprises 15-35% cholesterol, by weight or mol %.

In one embodiment, any amount of any compound recited herein in percentages (%) is weight % or mol. %.

In some embodiments, cholesterol refers to cholesterol and any derivatives thereof. Some non-limiting examples of cholesterol derivatives include: bile salts, steroid hormones, p-aminobenzoate of cholesterol, dihydrocholesterol, and hydroxycholesterol. One skilled in the art, will understand that a derivative of cholesterol will include any molecule that retains the cholesterol central molecule, but has additional side chains or groups added to it.

In some embodiments, the liposome comprises 5-10%, 5-15%. 5-20%, 1-10%, 1-15%, 1-20%, 5-9%, 10-15%, 12-18%, 12-20%, 10-20%, 5-8%, 5-7%, 5-6%, 6-10%, 6-9%, 6-8%, 6-7%, 7-10%, 7-9%, 7-8%, 8-10%, 8-9%, or 9-10% phosphatidyl phosphoric acid, by molarity or by weight. Each possibility represents a separate embodiment of the present invention. In some embodiments, 5-10%, 5-15%. 5-20%, 1-10%, 1-15%, 1-20%, 5-9%, 5-8%, 5-7%, 5-6%, 6-10%, 6-9%, 6-8%, 6-7%, 7-10%, 7-9%, 7-8%, 8-10%, 8-9%, 9-10%, 10-20%, 15-20%, 12-18%, 10-15%, or 15-20% of all the molecules of the liposome are phosphatidyl phosphoric acid. Each possibility represents a separate embodiment of the present invention.

In some embodiments, the liposome comprises 1-12%, 2-12%, 3-12%, 4-10%, 5-12%, 6-12%, 1-11%, 2-11%, 3-11%, 4-10%, 5-11%, 6-11%, 1-10%, 2-10%, 3-10%, 4-10%, 5-10%, 6-10%, 1-9%, 2-9%, 3-9%, 4-10%, 5-9%, 6-9%, 1-8%, 2-8%, 3-8%, 4-10%, 5-8%, 6-8%, 1-7%, 2-7%, 3-7%, 4-10%, 5-7%, 6-7%, 10-20%, 12-20%, 15-18%, 15-18%, or 15-20% phosphatidyl phosphoric acid, by weight or mol %. Each possibility represents a separate embodiment of the present invention. In some embodiments, the liposome comprises 3-7% phosphatidyl phosphoric acid, by weight.

In some embodiments, phosphatidyl phosphoric acid refers to phosphatidyl phosphoric acid and any derivatives thereof. One skilled in the art, will understand that a derivative of phosphatidyl phosphoric acid will include any molecule that retains the phosphatidyl phosphoric acid central molecule, but has additional side chains or groups added to it.

In some embodiments, the liposome comprises 20-60%, 30-60%, 40-60%, 50-60%, 20-50%, 30-50%, 40-50%, 40-70%, 50-70%, phosphatidyl choline, by molarity or weight. Each possibility represents a separate embodiment of the present invention. In some embodiments, 20-60%, 30-60%, 40-60%, 50-60%, 20-50%, 30-50%, 40-50%, 40-70%, 50-70%, of all the molecule of the liposome are phosphatidyl choline. Each possibility represents a separate embodiment of the present invention.

In some embodiments, the liposome comprises 45-80%, 50-80%, 55-80%, 60-80%, 65-80%, 45-75%, 50-75%, 55-75%, 60-75%, 65-75%, 45-70%, 50-70%, 55-70%, 60-70%, 65-70%, 45-65%, 50-65%, 55-65%, or 60-65% phosphatidyl choline, by weight or mol %. Each possibility represents a separate embodiment of the present invention. In some embodiments, the liposome comprises 50-75% phosphatidyl choline, by weight.

In some embodiments, phosphatidyl choline refers to phosphatidyl choline and any derivatives thereof. One skilled in the art, will understand that a derivative of phosphatidyl choline will include any molecule that retains the phosphatidyl choline central molecule, but has additional side chains or groups added to it.

In some embodiments, the liposome comprises a peptide anchored and conjugated to a succinate within the liposome bilayer, and wherein the peptide is on the outside of the liposome. In some embodiments, the succinate is 1,2-dioleoyl-sn-glycero-3-succinate. In some embodiments, the succinate passes completely through the lipid biolayer, such that the succinate extends to the outside of the liposome and to the interior of the liposome. In some embodiments, the succinate is entirely within the lipid bilayer, but the peptide is on the outside of the liposome. It will be well understood by one skilled in the art that the peptide must be on the outside of the liposome so that it can bind or interact with proteins that the liposome may encounter.

In some embodiments, the liposome comprises (by mol % or weight %) 0.1-2%, 0.1-1.8%, 0.1-1.6%, 0.1-1.4%, 0.1-1.2%, 0.1-1.0%, 0.1-0.8%, 0.3-2%, 0.3-1.8%, 0.3-1.6%, 0.3-1.4%, 0.3-1.2%, 0.3-1.0%, 0.3-0.8%, 0.5-2%, 0.5-1.8%, 0.5-1.6%, 0.5-1.4%, 0.5-1.2%, 0.5-1.0%, 0.5-0.8%, 0.7-2%, 0.7-1.8%, 0.7-1.6%, 0.7-1.4%, 0.7-1.2%, 0.7-1.0%, 0.7-0.8%, 0.9-2%, 0.9-1.8%, 0.9-1.6%, 0.9-1.4%, 0.9-1.2%, or 0.9-1.0% a peptide comprising the amino acid sequence HRERMS (SEQ ID NO: 1), conjugated and anchored to 1,2-dioleoyl-sn-glycero-3-succinate, by molarity. Each possibility represents a separate embodiment of the present invention. In some embodiments, 0.1-2%, 0.1-1.8%, 0.1-1.6%, 0.1-1.4%, 0.1-1.2%, 0.1-1.0%, 0.1-0.8%, 0.3-2%, 0.3-1.8%, 0.3-1.6%, 0.3-1.4%, 0.3-1.2%, 0.3-1.0%, 0.3-0.8%, 0.5-2%, 0.5-1.8%, 0.5-1.6%, 0.5-1.4%, 0.5-1.2%, 0.5-1.0%, 0.5-0.8%, 0.7-2%, 0.7-1.8%, 0.7-1.6%, 0.7-1.4%, 0.7-1.2%, 0.7-1.0%, 0.7-0.8%, 0.9-2%, 0.9-1.8%, 0.9-1.6%, 0.9-1.4%, 0.9-1.2%, or 0.9-1.0% (by mol % or weight %) of all the molecule of the liposome are a peptide comprising the amino acid sequence HRERMS (SEQ ID NO: 1), conjugated to 1,2-dioleoyl-sn-glycero-3-succinate, by molarity. Each possibility represents a separate embodiment of the present invention.

In some embodiments, the liposome comprises (by mol % or weight %) 0.054%, 0.05-3.5%, 0.05-3%, 0.05-2.5%, 0.05-2%, 0.05-1.5%, 0.05-1%, 0.1-4%, 0.1-3.5%, 0.1-3%, 0.1-2.5%, 0.1-2%, 0.1-1.5%, 0.1-1%, 0.25-4%, 0.25-3.5%, 0.25-3%, 0.25-2.5%, 0.25-2%, 0.25-1.5%, 0.25-1%, 0.5-4%, 0.5-3.5%, 0.5-3%, 0.5-2.5%, 0.5-2%, 0.5-1.5%, or 0.5-1%, a peptide comprising the amino acid sequence HRERMS (SEQ ID NO: 1), conjugated and anchored to 1,2-dioleoyl-sn-glycero-3-succinate, by weight. Each possibility represents a separate embodiment of the present invention. In some embodiments, the liposome comprises 0.1-3% a peptide comprising the amino acid sequence HRERMS (SEQ ID NO: 1), conjugated and anchored to 1,2-dioleoyl-sn-glycero-3-succinate, by weight.

In some embodiments, the peptide comprises at least one of the amino acid sequences selected from the group consisting of: HRERMS (SEQ ID NO: 1), RERMS (SEQ ID NO: 2), ARERMS (SEQ ID NO: 3), or AHRERMS (SEQ ID NO: 4). In some embodiments, the peptide comprises the amino acid sequence HRERMS (SEQ ID NO: 1). In some embodiments, the peptide consists of the amino acid sequence HRERMS (SEQ ID NO: 1). In some embodiments, the peptide is a targeting peptide. The term “targeting peptide” as used herein refers to a short amino acid sequence used to target the liposome to a specific tissue or location within the body of a subject. It will be well understood by one skilled in the art that the peptide must be on the outside of the liposome so that it can target the liposome.

In some embodiments, the peptide targets the liposome to a specific organ. In some embodiments, the peptide targets the liposome to specific regions within an organ. In some embodiments, the peptide targets the liposome to specific cells. In some embodiments, the peptide carries the liposome through the circulatory system to the target organ/region/cell. In some embodiments, the peptide targets the liposome to the brain. In some embodiments, the peptide allows the liposome to cross the blood brain barrier.

In another embodiment, a liposome of the present invention includes those composed primarily of vesicle-forming lipids. In another embodiment, a vesicle-forming lipid is a lipid that (a) can form spontaneously into bilayer vesicles in water, as exemplified by the phospholipids, or (b) is stably incorporated into lipid bilayers, with its hydrophobic moiety in contact with the interior, hydrophobic region of the bilayer membrane, and its head group moiety oriented toward the exterior, polar surface of the membrane.

In another embodiment, the vesicle-forming lipids are ones having two hydrocarbon chains, acyl chains, and a head group, either polar or nonpolar. In another embodiment, synthetic vesicle-forming lipids and naturally-occurring vesicle-forming lipids are utilized, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation.

In another embodiment, a liposome such as described herein is composed of natural phospholipids. In another embodiment, a liposome such as described herein is composed of mixed lipid chains with surfactant properties. In another embodiment, a liposome such as described herein is a multilamellar vesicle (MLV). In another embodiment, a liposome such as described herein is a small unilamellar vesicle (SUV). In another embodiment, a liposome such as described herein is a large unilamellar vesicle (LUV). In another embodiment, a liposome such as described herein is a cochleate vesicle.

In some embodiments, the liposome comprises cholesterol. In some embodiments, the liposome comprises a cholesterol derivative. In some embodiments, the cholesterol derivative is selected from the group consisting of: cholesterol pullulan and positively-charged cholesterol (e.g., DC-Chol).

In some embodiments, phosphatidyl choline includes naturally occurring, semi-synthetic or synthetic phosphatidylcholines (e.g., DSPC, DMPC, etc.). In some embodiments, the phosphatidylcholine is a non-naturally occurring phosphatidyl choline. In some embodiments, the phosphatidyl choline is an acyl phosphatidyl choline (e.g., DMPC, DPPC, POPC, DSPC, etc.). In some embodiments, phosphatidyl choline may be, for example, distearoyl phosphatidyl choline (DSPC), dimyristoyl phosphatidyl choline (DMPC), dipalmitoyl phosphatidyl choline (DPPC), palmitoyl oleoyl phosphatidyl choline (POPC), egg phosphatidyl choline (EPC), hydrogenated soya phosphatidylcholine (HSPC), etc. In some embodiments, the phosphatidyl choline is DMPC. In some embodiments, the phosphatidyl choline is DSPC. In some embodiments, the phosphatidyl choline is DPPC. In some embodiments, the phosphatidyl choline is POPC. In some embodiments, the phosphatidyl choline is EPC. In some embodiments, the phosphatidyl choline is HSPC.

In another embodiment, the present invention further comprises the use of derivatized lipids. Methods of preparing derivatized lipids and of forming polymer-coated liposomes are described in U.S. Pat. Nos. 5,013,556, 5,631,018 and 5,395,619, which are incorporated herein by reference in their entirety. In another embodiment, the hydrophilic polymer is stably coupled to the lipid, or coupled through an unstable linkage which may allow coated liposomes to shed the coating of polymer chains as they circulate in the bloodstream or in response to a stimulus.

In some embodiment, a liposome of the invention is prepared by a variety of techniques, such as those detailed in: U.S. Pat. Application 20150283078, Szoka, F., Jr., et al., Ann. Rev. Biophys. Bioeng. 9:467 (1980), U.S. Pat. Nos. 4,983,397; 6,476,068; 5,834,012; 5,756,069; 6,387,397; 5,534,241; 4,789,633; 4,925,661; 6,153,596; 6,057,299; 5,648,478; 6,723,338; 6,627218; U.S. Pat. App. Publication Nos: 2003/0224037; 2004/0022842; 2001/0033860; 2003/0072794; 2003/0082228; 2003/0212031; 2003/0203865; 2004/0142025; 2004/0071768; International Patent Applications WO 00/74646; WO 96/13250; WO 98/33481; Papahadjopolulos D, Allen T M, Gabizon A, et al. “Sterically stabilized liposomes. Improvements in pharmacokinetics and antitumor therapeutic efficacy” Proc Natl Acad Sci U.S.A. (1991) 88: 11460-11464; Allen T M, Martin F J. “Advantages of liposomal delivery systems for anthracyclines” Semin Oncol (2004) 31: 5-15 (suppl 13). Weissig et al. Pharm. Res. (1998) 15: 1552-1556, all of which are hereby incorporated by reference in their entireties.

In some embodiments, the liposome is lyophilized. In some embodiments, the liposome is sized. In some embodiments, the liposome's diameter is between 5nm to 300 nm. In another embodiment, the liposome's diameter is between 10 nm to 200 nm. In another embodiment, the liposome's diameter is between 50 nm to 200 nm. In another embodiment, the liposome's diameter is between 50 nm to 150 nm.

In one embodiment, a composition as described herein comprises a population of liposomes as described herein having a minimal size distribution. In one embodiment, at least 90% of the liposomes within a population of liposomes have a diameter selected from the range of 5 nm to 300 nm with a diameter size distribution of ±20%. In one embodiment, at least 95% of the liposomes within a population of liposomes have a diameter selected from the range of 5 nm to 300 nm with a diameter size distribution of ±20%. In one embodiment, at least 90% of the liposomes within a population of liposomes have a diameter selected from the range of 5 nm to 50 nm with a diameter size distribution of ±20%. In one embodiment, at least 95% of the liposomes within a population of liposomes have a diameter selected from the range of 5 nm to 50 nm with a diameter size distribution of ±20%. In one embodiment, at least 80% of the liposomes within a population of liposomes have a diameter of 10 nm with a diameter size distribution of ±20%. In one embodiment, at least 90% of the liposomes within a population of liposomes have a diameter of 10 nm with a diameter size distribution of ±20%. In one embodiment, at least 95% of the liposomes within a population of liposomes have a diameter of 10 nm with a diameter size distribution of ±20%.

In some embodiment, the zeta potential of a liposome of the invention is negative. In some embodiments, the zeta potential of a liposome of the invention is from −10 mV to −200 mV. In another embodiment, the zeta potential of a liposome of the invention is from −50 mV to −150 mV. In another embodiment, the zeta potential of a liposome of the invention is from −50 mV to −130 mV. In another embodiment, the zeta potential of a liposome of the invention is from −60 to −120 mV. In another embodiment, the zeta potential of a liposome of the invention is from −50 to −100 mV. In another embodiment, the zeta potential of a liposome of the invention is from −75 mV to −90 mV. In another embodiment, the zeta potential of a liposome of the invention is from −80 mV to −90 mV. In another embodiment, the zeta potential of a liposome of the invention is from −80 mV to −85 mV. In another embodiment, the zeta potential of a liposome of the invention is from −85 mV to −90 mV. In another embodiment, the zeta potential of a liposome of the invention is from −75 mV to −85 mV. In another embodiment, the zeta potential of a liposome of the invention is from −70 mV to −90 mV. In another embodiment, the zeta potential of a liposome of the invention is −75 mV, −80 mV, −85 mV, −83 mV, −90 mV, −100 mV, −120 mV.

The term “zeta potential” as used herein refers to the potential difference that exists between the surface of the liposome and fluid in which the liposome exists. In some embodiments, the fluid in which the liposome exists is saline. In some embodiments, the fluid is blood. In some embodiments, the fluid is cerebral spinal fluid. In some embodiments, the fluid is a pharmaceutically acceptable carrier.

As used herein, the terms “carrier” and “adjuvant” refer to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N. J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

In some embodiment, a process for the preparation of a liposome comprising a peptide comprises the steps of: (A) dissolving in an organic solvent such as chloroform: (1) Cholesterol (Ch), phosphatidyl phosphoric acid, and phosphatidyl choline; (B) adding to the mixture of step (A) a targeting peptide conjugated to a succinate such as but not limited to: 1,2-dioleoyl-sn-glycero-3-succinate or 1,2-dioleoyl-sn-glycero-3-succinate; (C) remove the organic solvent thus obtaining a dried lipid film; and (D) hydrating said dried lipid film, thereby obtaining a liposome comprising a targeting peptide.

In some embodiments, a liposome as described herein comprises at least 10% mol % or weight % Phosphatidyl phosphoric acid (PA). In some embodiments, a liposome as described herein comprises at least 12% mol % or weight % Phosphatidyl phosphoric acid (PA). In some embodiments, a liposome as described herein comprises at least 15% mol % or weight % Phosphatidyl phosphoric acid (PA). In some embodiments, a liposome as described herein comprises 10% to 20% (mol % or weight %) Phosphatidyl phosphoric acid (PA). In some embodiments, a liposome as described herein comprises 15% to 18% (mol % or weight %) Phosphatidyl phosphoric acid (PA). In some embodiments, a liposome as described herein comprises 15% to 20% (mol % or weight %) Phosphatidyl phosphoric acid (PA). In some embodiments, a liposome as described herein comprises up to 20% w/w or mol % of the liposome. In one embodiment, any value or range as described which is more than 10% and less than or equal to 20% w/w or mol % PA results in increase in both the stability and the selectivity/efficacy of the liposome. In one embodiment, any value or range as described which is more than 10% and less than or equal to 20% w/w or mol % PA results in maintaining/stabilizing the negative value of the zeta potential of the liposome.

In some embodiment, succinate is 1,2-dioleoyl-sn-glycero-3-succinate. In some embodiment, the liposome comprises 0.05-2%, 0.1-2%, 0.5-2%, 0.75-2%, 1-2%, 0.05-1.5%, 0.1-1.5%, 0.5-1.5%, 0.75-1.5%, 1-1.5%, 0.05-1%, 0.1-1%, 0.5-1%, 0.75-1%, 0.05-0.75%, 0.1-0.75%, 0.5-0.75%, 0.05-0.5%, or 0.1-0.5% succinate, by molarity. Each possibility represents a separate embodiment of the present invention.

In some embodiments, the liposome comprises 0.05-4%, 0.05-3.5%, 0.05-3%, 0.05-2.5%, 0.05-2%, 0.05-1.5%, 0.05-1%, 0.1-4%, 0.1-3.5%, 0.1-3%, 0.1-2.5%, 0.1-2%, 0.1-1.5%, 0.1-1%, 0.25-4%, 0.25-3.5%, 0.25-3%, 0.25-2.5%, 0.25-2%, 0.25-1.5%, 0.25-1%, 0.5-4%, 0.5-3.5%, 0.5-3%, 0.5-2.5%, 0.5-2%, 0.5-1.5%, or 0.5-1% succinate, by weight. Each possibility represents a separate embodiment of the present invention.

In another embodiment, hydrating is hydrating the dried lipid film in water. In another embodiment, hydrating is hydrating the dried lipid film in a buffer. In another embodiment, hydrating is hydrating the dried lipid film in an isotonic buffer. In another embodiment, hydrating is hydrating the dried lipid film in phosphate buffer saline.

In some embodiments, the targeting peptide comprises the amino acid sequence HRERMS (SEQ ID NO: 1), the succinate is 1,2-dioleoyl-sn-glycero-3-succinate and the peptide conjugated or anchored to 1,2-dioleoyl-sn-glycero-3-succinate comprises 0.1-2% of the liposome by molarity.

In some embodiments, the peptide HRERMS targets a liposome to the blood brain barrier (BBB). In some embodiments, the liposome comprising the HRERMS peptide is for use in transport across the BBB.

### Pharmaceutical Compositions

In some embodiments, the liposomes of the invention further comprise a drug or an imaging agent. In some embodiments, the drug is a nucleic acid molecule. In another embodiment, the drug is a ribozyme. In another embodiment, the drug is a peptide or a polypeptide. In another embodiment, the drug is a peptide nucleic acid. In another embodiment, the drug is a viral particle. In another embodiment, the drug is a chemical agent. In another embodiment, the drug is a cytokine. In another embodiment, the drug is a plasmid containing a gene and a suitable promoter for expression of the gene.

In one embodiment, the drug and/or an imaging agent is attached to or absorbed onto a lipid within the liposome. In one embodiment, the drug and/or an imaging agent is attached to, solubilized within or absorbed onto an aqueous or a polar moiety and/or compartment within the liposome.

In some embodiments, the drug is an anticancer agent. In some embodiments, the anticancer agent is a cytotoxic drug, including those known by skill in the art and medical practitioners. Exemplary anticancer agents include topoisomerase I inhibitors, vinca alkaloids, alkylating agents (including platinum compounds), taxanes and others known to those of skill in the art.

In some embodiments, the imaging agent is a dye. In some embodiments, the imaging agent is a contrast agent. In some embodiments, the imaging agent is a protein. In some embodiments, the imaging agent is a tagged molecule. In some embodiments, the imaging agent is radioactively tagged. In some embodiments, the imaging agent is fluorescently tagged. In some embodiments, the imaging agent is magnetically tagged.

In another embodiment, the liposome comprises a single drug. In another embodiment, the liposome comprises more than one drug. In another embodiment, the liposome comprises a combination therapy. In some embodiments, the liposome comprises a single imagining agent. In some embodiments, the liposome comprises more than one imaging agent.

In another embodiment, the drug as described herein comprises an alkaloid, an alkylating agent, an anti-tumor antibiotic, an antimetabolite, a hormone and hormone analog, immunomodulator, photosensitizing agent, antibody, peptide, anti-mitotic agent, or any combination thereof. Each possibility represents a separate embodiment of the present invention. In another embodiment, the drug as described herein comprises a plant alkaloid.

In some embodiments, the drug is a chemotherapeutic agent. Chemotherapeutic agents will be well known to one skilled in the art, but a non-limiting list includes: cyclophosphamide, mechlorethamine, chlorambucil, melphalan, doxorubicin, dacarbazine, nitrosoureas, temozolomide (TMZ), daunorubicin, epirubicin, idarubicin, mitoxantrone, valrubicin, paclitaxel, docetaxel, abraxane, taxotere, varinostat, romidepsin, irinotecan, topotecan, etoposide, teniposide, tafluposide, bortezomib, erlotinib, getitinib, imatinib, vermurafenib, vismodegib, azacytidine, azathioprine, capecitabine, cytarabine, doxifluridine, fluorouracil, gemcitabine, hydroxyurea, mercaptopurine, methotrexate, tioguanine, bleomycin, actinomycin, carboplatin, cisplatin, oxaliplatin, tretinoin, alitretinoin, bexarotene, vinblastine, vincristine, vindesine, and vinorelbine.

In some embodiments, the drug is selected from the group consisting of: curcumin and TMZ. In some embodiments, the biological agent is curcumin. In some embodiments, the biological agent is TMZ.

In some embodiments, the drug or imaging agent is hydrophilic and in an aqueous solution. In some embodiments, the drug or imaging agent is in an isotonic buffer. In some embodiments, the drug or imaging agent is in an aqueous organic solvent. In some embodiments, the drug or imaging agent is in an alcohol. In some embodiments, the drug or imaging agent is in methanol or methanol/chloroform. In some embodiments, the drug or imaging agent is hydrophilic and encapsulated by the liposome.

As used herein, the term “hydrophilic” refers to a molecule that is attracted to water, dissolves in water and whose interaction with water is thermodynamically favorable. In some embodiments, hydrophilic molecules have a solubility of at least 10, 20, 30, 40, 50, 60, 70, 80, 90, 100, 200, 300, 400, 500, or 1000 mg/ml in water or other polar solvents. Each possibility represents a separate embodiment of the present invention. The core of the liposome is hydrophilic, thus in some embodiments, the liposome will encapsulate hydrophilic molecules.

In some embodiments, the drug or imaging agent in hydrophobic and in a non-aqueous solution. In some embodiments, the drug or imaging agent is in a non-aqueous organic solvent. In some embodiments, the drug or imaging agent is in acetone. In some embodiments, the drug or imaging agent is in acetonitrile. In some embodiments, the drug or imaging agent is in methanol/chloroform. In some embodiments, the drug or imaging agent is hydrophobic and embedded in the lipid layer of the liposome. In some embodiments, the drug or imaging agent is hydrophobic and embedded in the lipid bilayer of the liposome. In some embodiments, the hydrophobic drug or imaging agent is between the two layers of the lipid bilayer. In some embodiments, the hydrophobic drug is between individual lipid molecules of one layer of the lipid layer.

As used herein, the term “hydrophobic” refers to a molecule that is repelled by water, does not dissolves in water and whose interaction with water is thermodynamically unfavorable. In some embodiments, a hydrophobic molecule is lipid soluble. In some embodiments, hydrophobic molecules have a solubility of no greater than 25, 20, 15, 10, 5, 1, 0.8, 0.6, 0.4, 0.2, 0.1, 0.05, or 0.01 mg/ml in water or other polar solvents. Each possibility represents a separate embodiment of the present invention. The lipid layer or bilayer of a liposome is hydrophobic, thus in some embodiments the drug or imaging agent is embedded in the lipid layer of the liposome.

In another embodiment, the process for obtaining a liposome as described herein further comprises loading a lipid soluble drug onto or into a liposome. In another embodiment, the process for obtaining a liposome as described herein further comprises loading a lipid soluble drug onto or into a liposome by: (1) heating “a loading composition” comprising the lipid soluble drug and the liposome to a temperature that is 0.01° C. to 5° C. above the phase-inversion temperature of the lipids of the liposome thus obtaining a water in oil emulsion; (2) cooling the loading composition to a temperature that is 0.01° C. to 10° C. below the phase-inversion temperature thus obtaining an oil in water emulsion. In another embodiment, the above process for loading a lipid soluble drug onto or into a liposome includes at least one repetition of steps (1) and (2) thus forming at least one cycle. In another embodiment, a cycle is repeated at least twice. In another embodiment, a cycle is repeated 2-10 times. In another embodiment, a cycle is repeated 2-5 times.

In another embodiment, cooling is adding cold water. In another embodiment, cooling is refrigerating. In another embodiment, heating and cooling are performed at a rate of 0.5° C. to 20° C./min. In another embodiment, heating and cooling are performed at a rate of 1° C. to 10° C./min. In another embodiment, heating and cooling are performed at a rate of 1° C. to 5° C./min. In another embodiment, heating and cooling are performed at a rate of 2° C. to 8° C./min. In another embodiment, heating and cooling are performed at a rate of 5° C. to 10° C./min.

In another embodiment, heating is heating to a temperature of 60° C. to 100° C. In another embodiment, heating is heating to a temperature of 75° C. to 95° C. In another embodiment, heating is heating to a temperature of 75° C. to 85° C. In another embodiment, heating is heating to a temperature of 80° C. to 90° C. In another embodiment, heating is heating to a temperature of 90° C. to 100° C.

In another embodiment, cooling is cooling to a temperature of 40° C. to 80° C. In another embodiment, cooling is cooling to a temperature of 50° C. to 60° C. In another embodiment, cooling is cooling to a temperature of 60° C. to 70° C. In another embodiment, cooling is cooling to a temperature which is at least 5° C. below the maximal heating temperature. In another embodiment, cooling is cooling to a temperature which is at least 10° C. below the maximal heating temperature. In another embodiment, cooling is cooling to a temperature which is at least 15° C. below the maximal heating temperature. In another embodiment, cooling is cooling to a temperature which is at least 20° C. below the maximal heating temperature.

In another embodiment, the present invention provides a liposome comprising a drug or imaging agent obtained by the processes described above.

In some embodiments, the drug is a central nervous system (CNS) drug. A central nervous system drug, is a drug that treats or is suspected of treating a disease of the central nervous system. Diseases of the central nervous system will be well known to one skilled in the art, and include diseases of the brain, and diseases of the spinal cord. In some embodiments, the disease of the central nervous system is selected from the group consisting of: brain cancer, Parkinson's disease, Huntington's disease, and Alzheimer's disease. In some embodiments, the brain cancer is glioblastoma multiform, herein referred to as glioblastoma. In some embodiments, the CNS drug is selected form the group consisting of: a brain cancer therapeutic, a Parkinson's disease therapeutic, a Huntington's disease therapeutic, and an Alzheimer's disease therapeutic. In some embodiments, the brain cancer is glioblastoma multiform, herein referred to as glioblastoma.

In another embodiment, the liposome of the present invention enables the delivery of a drug such as an anticancer agent without the induction of devastating side effects produced by the drug. In some embodiments, the liposomes of the present invention stabilize the drug. In some embodiments, the liposomes of the present invention reduce the dosing of the drug. In some embodiments, the liposomes of the invention are loaded with doses of the drug that are below the standard dose of the drug. In some embodiments, the liposomes of the invention reduce delivery of the drug to cells or tissues that are not the target of the drug. In some embodiments, the liposomes of the invention reduce a side effect of the drug. Thus, the present invention bypasses the drawbacks and toxicity of most anticancer agents which often have a relatively small range of therapeutic index, (i.e., the narrow dosage range in which cancer cells are destroyed without unacceptable toxicity to the individual). In some embodiments, the liposomes of the invention broaden the range of the therapeutic index.

In another embodiment, the liposome of the present invention carries, encapsulates, or is embedded with the drug or imaging agent as described herein, stabilizes it, penetrates through the BBB, and unloads the drug or imaging agent in the brain. In some embodiments, the liposome unloads the drug or imaging agent at a precise predetermined location or cell type (such as a cancer cell). In another embodiment, the liposome of the present invention carries the drug or imaging agent as described herein to cancerous cells. In another embodiment, the safety and specificity profile of the liposome of the present invention which carries an anti-cancer agent brings about the reduction of common side effects such as nausea and vomiting. In another embodiment, this specificity allows the use of highly toxic compounds to be delivered to pre-determined sites without unintended leakages of these toxic compounds.

Thus, the liposome carrier described herein, in some embodiments, reduces side effects common to a wide range of anticancer agents which include: hair loss (alopecia); appetite loss; weight loss; taste changes; stomatitis and esophagitis (inflammation and sores); constipation; diarrhea; fatigue; heart damage; nervous system changes; lung damage; reproductive tissue damage; liver damage; kidney and urinary system damage.

In some embodiments, the liposome comprises 0.1-2, 0.3-2, 0.5-2, 0.7-2, 0.9-2, 1-2, 1.1-2, 1.3-2, 1.5-2, 1.7-2, 1.9-2, 0.1-1.9, 0.3-1.9, 0.5-1.9, 0.7-1.9, 0.9-1.9, 1-1.9, 1.1-1.9, 1.3-1.9, 1.5-1.9, 1.7-1.9, 0.1-1.7, 0.3-1.7, 0.5-1.7, 0.7-1.7, 0.9-1.7, 1-1.7, 1.1-1.7, 1.3-1.7, 1.5-1.7, 0.1-1.5, 0.3-1.5, 0.5-1.5, 0.7-1.5, 0.9-1.5, 1-1.5, 1.1-1.5, 1.3-1.5, 0.1-1.3, 0.3-1.3, 0.5-1.3, 0.7-1.3, 0.9-1.3, 1-1.3, 1.1-1.3, 0.1-1.1, 0.3-1.1, 0.5-1.1, 0.7-1.1, 0.9-1.1, 1-1.1, 0.1-1, 0.3-1, 0.5-1, 0.7-1, 0.9-1, 0.1-0.9, 0.3-0.9, 0.5-0.9, 0.7-0.9, 0.1-0.7, 0.3-0.7, 0.5-0.7, 0.1-0.5, 0.3-0.5, or 0.1-0.3% drug, by molarity. Each possibility represents a separate embodiment of the invention. In some embodiments, the liposome comprises 0.1-1.8% drug, by molarity.

In some embodiments, the liposome comprises 0.1-10, 0.3-10, 0.5-10, 0.7-10, 0.9-10, 1-10, 1.1-10, 1.3-10, 1.5-10, 1.7-10, 1.9-10, 0.1-9, 0.3-9, 0.5-9, 0.7-9, 0.9-9, 1-9, 1.1-9, 1.3-9, 1.5-9, 1.7-9, 1.9-9, 0.1-8, 0.3-8, 0.5-8, 0.7-8, 0.9-8, 1-8, 1.1-8, 1.3-8, 1.5-8, 1.7-8, 1.9-8, 0.1-7, 0.3-7, 0.5-7, 0.7-7, 0.9-7, 1-7, 1.1-7, 1.3-7, 1.5-7, 1.7-7, 1.9-7, 0.1-6, 0.3-6, 0.5-6, 0.7-6, 0.9-6, 1-6, 1.1-6, 1.3-6, 1.5-6, 1.7-6, 1.9-6, 0.1-5, 0.3-5, 0.5-5, 0.7-5, 0.9-5, 1-5, 1.1-5, 1.3-5, 1.5-5, 1.7-5, 1.9-5, 0.1-4, 0.3-4, 0.5-4, 0.7-4, 0.9-4, 1-4, 1.1-4, 1.3-4, 1.5-4, 1.7-4, 1.9-4, 0.1-3, 0.3-3, 0.5-3, 0.7-3, 0.9-3, 1-3, 1.1-3, 1.3-3, 1.5-3, 1.7-3, 1.9-3, 0.1-2, 0.3-2, 0.5-2, 0.7-2, 0.9-2, 1-2, 1.1-2, 1.3-2, 1.5-2, 1.7-2, 1.9-2, 0.1-1.9, 0.3-1.9, 0.5-1.9, 0.7-1.9, 0.9-1.9, 1-1.9, 1.1-1.9, 1.3-1.9, 1.5-1.9, 1.7-1.9, 0.1-1.7, 0.3-1.7, 0.5-1.7, 0.7-1.7, 0.9-1.7, 1-1.7, 1.1-1.7, 1.3-1.7, 1.5-1.7, 0.1-1.5, 0.3-1.5, 0.5-1.5, 0.7-1.5, 0.9-1.5, 1-1.5, 1.1-1.5, 1.3-1.5, 0.1-1.3, 0.3-1.3, 0.5-1.3, 0.7-1.3, 0.9-1.3, 1-1.3, 1.1-1.3, 0.1-1.1, 0.3-1.1, 0.5-1.1, 0.7-1.1, 0.9-1.1, 1-1.1, 0.1-1, 0.3-1, 0.5-1, 0.7-1, 0.9-1, 0.1-0.9, 0.3-0.9, 0.5-0.9, 0.7-0.9, 0.1-0.7, 0.3-0.7, 0.5-0.7, 0.1-0.5, 0.3-0.5, or 0.1-0.3% imaging agent, by molarity, by mol % or by weight %. Each possibility represents a separate embodiment of the invention. In some embodiments, the liposome comprises 0.1-10% imaging agent, by molarity.

In some embodiments, the liposome comprises 0.1-10, 0.3-10, 0.5-10, 0.7-10, 0.9-10, 1-10, 1.1-10, 1.3-10, 1.5-10, 1.7-10, 1.9-10, 0.1-9, 0.3-9, 0.5-9, 0.7-9, 0.9-9, 1-9, 1.1-9, 1.3-9, 1.5-9, 1.7-9, 1.9-9, 0.1-8, 0.3-8, 0.5-8, 0.7-8, 0.9-8, 1-8, 1.1-8, 1.3-8, 1.5-8, 1.7-8, 1.9-8, 0.1-7, 0.3-7, 0.5-7, 0.7-7, 0.9-7, 1-7, 1.1-7, 1.3-7, 1.5-7, 1.7-7, 1.9-7, 0.1-6, 0.3-6, 0.5-6, 0.7-6, 0.9-6, 1-6, 1.1-6, 1.3-6, 1.5-6, 1.7-6, 1.9-6, 0.1-5, 0.3-5, 0.5-5, 0.7-5, 0.9-5, 1-5, 1.1-5, 1.3-5, 1.5-5, 1.7-5, 1.9-5, 0.1-4, 0.3-4, 0.5-4, 0.7-4, 0.9-4, 1-4, 1.1-4, 1.3-4, 1.5-4, 1.7-4, 1.9-4, 0.1-3, 0.3-3, 0.5-3, 0.7-3, 0.9-3, 1-3, 1.1-3, 1.3-3, 1.5-3, 1.7-3, 1.9-3, 0.1-2, 0.3-2, 0.5-2, 0.7-2, 0.9-2, 1-2, 1.1-2, 1.3-2, 1.5-2, 1.7-2, 1.9-2, 0.1-1.9, 0.3-1.9, 0.5-1.9, 0.7-1.9, 0.9-1.9, 1-1.9, 1.1-1.9, 1.3-1.9, 1.5-1.9, 1.7-1.9, 0.1-1.7, 0.3-1.7, 0.5-1.7, 0.7-1.7, 0.9-1.7, 1-1.7, 1.1-1.7, 1.3-1.7, 1.5-1.7, 0.1-1.5, 0.3-1.5, 0.5-1.5, 0.7-1.5, 0.9-1.5, 1-1.5, 1.1-1.5, 1.3-1.5, 0.1-1.3, 0.3-1.3, 0.5-1.3, 0.7-1.3, 0.9-1.3, 1-1.3, 1.1-1.3, 0.1-1.1, 0.3-1.1, 0.5-1.1, 0.7-1.1, 0.9-1.1, 1-1.1, 0.1-1, 0.3-1, 0.5-1, 0.7-1, 0.9-1, 0.1-0.9, 0.3-0.9, 0.5-0.9, 0.7-0.9, 0.1-0.7, 0.3-0.7, 0.5-0.7, 0.1-0.5, 0.3-0.5, or 0.1-0.3% drug or imaging agent, by molarity, by mol % or by weight %. Each possibility represents a separate embodiment of the invention. In some embodiments, the liposome comprises 0.1-10% drug or imaging agent, by molarity.

In some embodiments, the liposome comprises 0.01-5%, 0.01-4.5%, 0.01-4%, 0.01-3.5%, 0.01-3%, 0.01-2.5%, 0.01-2%, 0.01-1.5%, 0.01-1%, 0.05-5%, 0.05-4.5%, 0.05-4%, 0.05-3.5%, 0.05-3%, 0.05-2.5%, 0.05-2%, 0.05-1.5%, 0.05-1%, 0.1-5%, 0.1-4.5%, 0.1-4%, 0.1-3.5%, 0.1-3%, 0.1-2.5%, 0.1-2%, 0.1-1.5%, 0.1-1%, 0.15-5%, 0.15-4.5%, 0.15-4%, 0.15-3.5%, 0.15-3%, 0.15-2.5%, 0.15-2%, 0.15-1.5%, 0.15-1%, 0.2-5%, 0.2-4.5%, 0.2-4%, 0.2-3.5%, 0.2-3%, 0.2-2.5%, 0.2-2%, 0.2-1.5%, 0.2-1%, 0.25-5%, 0.25-4.5%, 0.25-4%, 0.25-3.5%, 0.25-3%, 0.25-2.5%, 0.25-2%, 0.25-1.5%, 0.25-1%, 0.3-5%, 0.3-4.5%, 0.3-4%, 0.3-3.5%, 0.3-3%, 0.3-2.5%, 0.3-2%, 0.3-1.5%, 0.3-1%, 0.35-5%, 0.35-4.5%, 0.35-4%, 0.35-3.5%, 0.35-3%, 0.35-2.5%, 0.35-2%, 0.35-1.5%, 0.35-1%, 0.4-5%, 0.4-4.5%, 0.4-4%, 0.4-3.5%, 0.4-3%, 0.4-2.5%, 0.4-2%, 0.4-1.5%, 0.4-1%, 0.45-5%, 0.45-4.5%, 0.45-4%, 0.45-3.5%, 0.45-3%, 0.45-2.5%, 0.45-2%, 0.45-1.5%, 0.45-1%, 0.5-5%, 0.5-4.5%, 0.5-4%, 0.5-3.5%, 0.5-3%, 0.5-2.5%, 0.5-2%, 0.5-1.5%, or 0.5-1%, drug, by weight, by mol % or by weight %. Each possibility represents a separate embodiment of the invention. In some embodiments, the liposome comprises 0.1-1.8% drug, by weight.

In some embodiments, the liposome comprises 0.01-12.5%, 0.01-10%, 0.01-7.5%, 0.01-5%, 0.01-4.5%, 0.01-4%, 0.01-3.5%, 0.01-3%, 0.01-2.5%, 0.01-2%, 0.01-1.5%, 0.01-1%,0.05-12.5%, 0.05-10%, 0.05-7.5%, 0.05-5%, 0.05-4.5%, 0.05-4%, 0.05-3.5%, 0.05-3%, 0.05-2.5%, 0.05-2%, 0.05-1.5%, 0.05-1%, 0.1-12.5%, 0.1-10%, 0.1-7.5%, 0.1-5%, 0.1-4.5%, 0.1-4%, 0.1-3.5%, 0.1-3%, 0.1-2.5%, 0.1-2%, 0.1-1.5%, 0.1-1%, 0.2-12.5%, 0.2-10%, 0.2-7.5%, 0.2-5%, 0.2-4.5%, 0.2-4%, 0.2-3.5%, 0.2-3%, 0.2-2.5%, 0.2-2%, 0.2-1.5%, 0.2-1%, 0.3-12.5%, 0.3-10%, 0.3-7.5%, 0.3-5%, 0.3-4.5%, 0.3-4%, 0.3-3.5%, 0.3-3%, 0.3-2.5%, 0.3-2%, 0.3-1.5%, 0.3-1%, 0.4-12.5%, 0.4-10%, 0.4-7.5%, 0.4-5%, 0.4-4.5%, 0.4-4%, 0.4-3.5%, 0.4-3%, 0.4-2.5%, 0.4-2%, 0.4-1.5%, 0.4-1%, 0.5-12.5%, 0.5-10%, 0.5-7.5%, 0.5-5%, 0.5-4.5%, 0.5-4%, 0.5-3.5%, 0.5-3%, 0.5-2.5%, 0.5-2%, 0.5-1.5%, or 0.5-1% imaging agent, by weight, by mol % or by weight %. Each possibility represents a separate embodiment of the invention. In some embodiments, the liposome comprises 0.01-10% imagine agent, by weight.

In some embodiments, the liposome comprises 0.01-12.5%, 0.01-10%, 0.01-7.5%, 0.01-5%, 0.01-4.5%, 0.01-4%, 0.01-3.5%, 0.01-3%, 0.01-2.5%, 0.01-2%, 0.01-1.5%, 0.01-1%,0.05-12.5%, 0.05-10%, 0.05-7.5%, 0.05-5%, 0.05-4.5%, 0.05-4%, 0.05-3.5%, 0.05-3%, 0.05-2.5%, 0.05-2%, 0.05-1.5%, 0.05-1%, 0.1-12.5%, 0.1-10%, 0.1-7.5%, 0.1-5%, 0.1-4.5%, 0.1-4%, 0.1-3.5%, 0.1-3%, 0.1-2.5%, 0.1-2%, 0.1-1.5%, 0.1-1%, 0.2-12.5%, 0.2-10%, 0.2-7.5%, 0.2-5%, 0.2-4.5%, 0.2-4%, 0.2-3.5%, 0.2-3%, 0.2-2.5%, 0.2-2%, 0.2-1.5%, 0.2-1%, 0.3-12.5%, 0.3-10%, 0.3-7.5%, 0.3-5%, 0.3-4.5%, 0.3-4%, 0.3-3.5%, 0.3-3%, 0.3-2.5%, 0.3-2%, 0.3-1.5%, 0.3-1%, 0.4-12.5%, 0.4-10%, 0.4-7.5%, 0.4-5%, 0.4-4.5%, 0.4-4%, 0.4-3.5%, 0.4-3%, 0.4-2.5%, 0.4-2%, 0.4-1.5%, 0.4-1%, 0.5-12.5%, 0.5-10%, 0.5-7.5%, 0.5-5%, 0.5-4.5%, 0.5-4%, 0.5-3.5%, 0.5-3%, 0.5-2.5%, 0.5-2%, 0.5-1.5%, or 0.5-1°% drug or imaging agent, by weight, by mol % or by weight %. Each possibility represents a separate embodiment of the invention. In some embodiments, the liposome comprises 0.01-10% drug or imagine agent, by weight.

In some embodiments, the composition comprises liposomes comprising a dose of drug that is below the standard dose for a particular condition or disease. In some embodiments, the liposomes comprise 50, 40, 30, 20, 10, 5, 1, 0.5, or 0.1% of the standard dose of the drug for a particular condition or disease. Each possibility represents a separate embodiment of the invention. For example, the standard dose of TMZ for glioblastoma multiform is 75 mg/m² (Brock, et al., 1998, Cancer Research, 58: 4363-67, http://reference.medscape.com/drug/temodartemozolomide-342229). In some embodiments, the liposomes comprise TMZ at a dose of 0.1-40, 0.1-35, 0.1-30, 0.1-25, 0.1-20, 0.1-15, 0.1-10, 0.1-5, 0.5-40, 0.5-35, 0.5-30, 0.5-25, 0.5-20, 0.5-15, 0.5-10, 0.5-5, 1-40, 1-35, 1-30, 1-25, 1-20, 1-15, 1-10, 1-5, 5-40, 5-35, 5-30, 5-25, 5-20, 5-15, or 5-10 mg/m². Each possibility represents a separate embodiment of the present invention. In some embodiments, the composition comprises liposomes comprises TMZ at a dose of 0.1-40 mg/m².

Standard doses for drugs and imaging agents can be found by one skilled in the art on several medication websites such as www.medscape.com, or www.drugs.com, or by examining the dosing data provided by the drug manufacturer. The standard dose may vary for a drug depending on the condition being treated.

By another aspect, the present invention provides a pharmaceutical composition comprising the liposomes of the invention and a pharmaceutically acceptable carrier, adjuvant or excipient.

As used herein, the terms “carrier”, “excipient” and “adjuvant” refer to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N. J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Pharmaceutical Compositions and Therapeutic Use

By another aspect, there is provided a method of treating or ameliorating a brain disease in a subject in need thereof, the method comprising: administering the pharmaceutical compositions of the present invention to the subject, thereby treating the brain disease.

By another aspect, there is provided a method for prolonging the half-life of a drug or imaging agent in a body of a subject, the method comprising: administering the pharmaceutical compositions of the present invention to the subject, thereby prolonging the half-life of a drug or imaging agent in the body of a subject.

A subject in need of treatment for a brain disease includes, but is not limited to, a subject with a terminal brain disease, a subject with a degenerative brain disease, a subject who is already receiving treatment for a brain disease, and a subject who is not receiving treatment for the brain disease.

In some embodiments, the brain disease for which the subject is in need of treatment is selected from the group consisting of brain cancer, Parkinson's disease, Huntington's disease, and Alzheimer's disease. In some embodiments, the brain cancer is glioblastoma.

In some embodiments, the pharmaceutical composition comprises the drug or imaging agent, in a dose significantly lower than is the medically accepted dose for treating a brain disease. In some embodiments, the dose of the pharmaceutical composition is 0.1%, 0.5%, 1%, 5%, 10%, 20%, 30%, 40%, 50% of the medically accepted dose for treating a brain disease. Each possibility represents a separate embodiment of the present invention. In some embodiments, the dose of the pharmaceutical composition is 1/40^{th}, 1/35^{th}, 1/30^{th}, 1/25^{th}, 1/20^{th}, 1/15^{th}, 1/10^{th} of the medically accepted dose for treating a brain disease. Each possibility represents a separate embodiment of the present invention. In some embodiments, the dose of the pharmaceutical composition is 1/30^{th} of the medically accepted dose for treating a brain disease.

In some embodiments, the dose of the drug or imaging agent is 0.1-75, 0.5-75, 1-75, 5-75, 10-75, 15-75, 0.1-50, 0.5-50, 1-50, 5-50, 10-50, 15-50, 0.1-40, 0.5-40, 1-40, 5-40, 10-40, 15-40, 0.1-40, 0.5-40, 1-40, 5-40, 10-40, 15-40, 0.1-30, 0.5-30, 1-30, 5-30, 10-30, 15-30, 0.1-30, 0.5-30, 1-30, 5-30, 10-30, 15-30, 0.1-25, 0.5-25, 1-25, 5-25, 10-25, 15-25, 0.1-25, 0.5-25, 1-25, 5-25, 10-25, 15-25, 0.1-20, 0.5-20, 1-20, 5-20, 10-20, 15-20, 0.1-15, 0.5-15, 1-15, 5-15, 10-15, 0.1-10, 0.5-10, 1-10, 5-10, 0.1-7.5, 0.5-7.5, 1-7.5, 5-7.5, 0.1-5, 0.5-5, 1-5 mg/m². Each possibility represents a separate embodiment of the present invention. In some embodiments, the pharmaceutical composition comprises TMZ, and the TMZ is present in a dose of 0.1-40 mg/m².

Dosing can be calculated by two different methods of determining the body size of the patient. The first method is weighing the patient, and in such a case the dose is mg/kg of the patient. The second method is to measure the body surface area of the patient, and in such a case the dose is mg/m² of the patient. The second method is generally considered preferable for anti-cancer treatments. In mice, the mg/kg dose can be converted to an approximate mg/m² dose by multiplying by 3. In humans, the mg/kg dose can be converted to an approximate mg/m² dose by multiplying by 37. A mouse dosage can be converted to an approximate human dosage by dividing the mouse dose by 12.3.

In some embodiments, the method of treating or ameliorating a brain disease further comprises administering the best practice therapy for the brain disease to the subject. The term “best practice” as used herein, refers to the treatment for a certain disease that is widely used by healthcare professionals and is accepted by medical experts as the proper treatment. The best practice therapy is the therapy that medical professionals have accepted as being the most correct or most effective in treating a disease. In some embodiments, there is more than one best practice therapy.

In some embodiments, the disease in brain cancer and the best practice therapy is cancer therapy. In some embodiments, the cancer therapy is selected from the group consisting of: radiation therapy, and chemotherapy.

In some embodiments, prolonging the half-life of a drug or imaging agent comprises extending the half-life by at least 10%, 20%, 30%, 40%, 50%, 60%, 70%, 80%, 90%, 100%, 150%, 200%, 250%, 300%, 350%, 400%, 450%, or 500%. Each possibility represents a separate embodiment of the invention. In some embodiments, extending the half-life is extending the half-life in the bloodstream. In some embodiments, extending the half-life is extending the half-life in cells. In some embodiments, extending the half-life of a drug or imaging agent extends the time between doses of a drug or imaging agent.

### EXAMPLES

### Example 1: Design of Liposomes for Delivery of Curcumin

In recent years, new drug targets have been identified and potential drug molecules synthesized and analyzed for efficacy. Some of those molecules are large and lipophilic. The improvement of solubility and dissolution profiles of these lipophilic drug molecules, without altering the molecular structure, is a particular challenge for the successful development of pharmaceutical products. One possible carrier for these drugs is the liposome, but integration of high levels of these drugs into the liposome is also a challenge.

In an attempt to formulate a new efficient carrier, carriers found in nature were used as a model for a universal lipid carrier that would have high solubility, delivery through the blood and dissolution at the target. The carrier would need to be flexible, biodegradable and targetable to specific tissues. The specific targeting mechanism employed was a fusion protein containing a six-amino acid peptide (HRERMS) from amyloid beta, covalently conjugated to 1,2-dioleoyl-sn-glycero-3-succinate. This fusion protein targets the liposome to the blood brain barrier (BBB) and allows for rapid and effective transport of the liposome across the barrier to the brain.

To test various liposome compositions, the water insoluble drug curcumin was employed as the therapeutic agent to be incorporated into the liposome. Curcumin, has been used for many clinical purposes, including as an antioxidant, antibacterial, anti-inflammatory agent, anti-neurodegenerative processes and anticancer agent. Curcumin however, is highly water-insoluble, has a very short biological half-life, and poor pharmacokinetics. Previous attempts at liposomes for the delivery of curcumin comprised cholesterol (Ch), 1,2-dioleyl-sn-glycero-3-phosphocholine (DOPC), 1,2-dioleyl-sn-glycero-3-phosphoethanolamine (DOPE), and 1,2-dioleoyl-sn-glycero-3-[phosphor-L-serine] (DOPS), usually in equimolar amounts.

In order to decrease aggregation of liposomes and to increase carrier specificity a negative Zeta potential in the liposome is greatly advantageous. A negative Zeta (below −40 mV ideally) results in liposomes repelling one another, and decreases aggregation. Further, positively or neutrally charged liposomes will nonspecifically bind to cells in the blood or tissues. Lastly, a negative Zeta potential enables increased conjugating of the β-amyloid peptides, or any other targeting peptide, aimed at giving the liposomes tissue targeting.

Various compositions of liposomes for delivery of curcumin were tested and evaluated for drug loading, drug stability and half-life, and effective targeting in the body. Over all, the most effective liposome composition found, comprised the following: 30-50% cholesterol (Ch) by molar ratio, mol % or weight %, 5-20% phosphatidyl phosphoric acid (PA) by molar ratio, mol % or weight %, and 40-6% phosphatidyl choline (PC) by molar ratio, mol % or weight %. The PA was found to be essential for maintenance of the Zeta potential below −40Mv. The targeting peptide was dissolved in chloroform/methanol and added to the mixture at 0.1-2% by molar ratio, mol % or weight %.

Loading of the drug into the liposome was achieved by pre-dissolving the target drug during the preparation of the liposome, thereby enabling a desired and reproducible concentration of the drug in the most stabilized environment. A concentration of drug at a molar ratio, mol % or weight % between 0.1-1.5% was found to be optimal. Utilization of this formulation improved solubility of curcumin by more than 100 times and also served to protect the drug from degradation and prolong its biological half-life.

### Example 2: Drug Delivery in a Mouse Model of Glioblastoma

In order to assess the efficacy of the liposome composition in delivering a therapeutic agent to the brain, glioblastoma in a mouse model was employed. Curcumin was integrated into liposomes comprising cholesterol: phosphatidyl phosphoric acid: phosphatidyl choline in molar ratios of 35:7:58. The targeting peptide was present at 0.5% by molar ratio, mol % or weight %, and the curcumin was at a final concentration of 1% by molar ratio, mol % or weight %. The resulting liposomes where lyophilized, hydrated and downsized into a diameter of 0.1 μm±20%.

As a positive control, Temozolomide (TMZ), a highly potent anti-cancer alkylating agent, with moderate solubility in water, was also dissolved in carrier liposomes. These liposomes comprised cholesterol: phosphatidyl phosphoric acid: phosphatidyl choline in molar ratios of 40:5:55. The molar concentration of the drug and targeting peptide were unchanged.

As a model of glioblastoma, SCID immuno-deficient mice were implanted with U87 glioblastoma cells. The U87 cells had previously been transfected with a luciferase gene, such that upon intraperitoneally injection of the mice with luciferin, the cells would fluoresce, allowing for accurate quantification and imaging of the tumor using the IVIS 200 imaging system. The degree of fluorescence measured was proportional to the number of cancer cells, and therefore indicated the cancer growth.

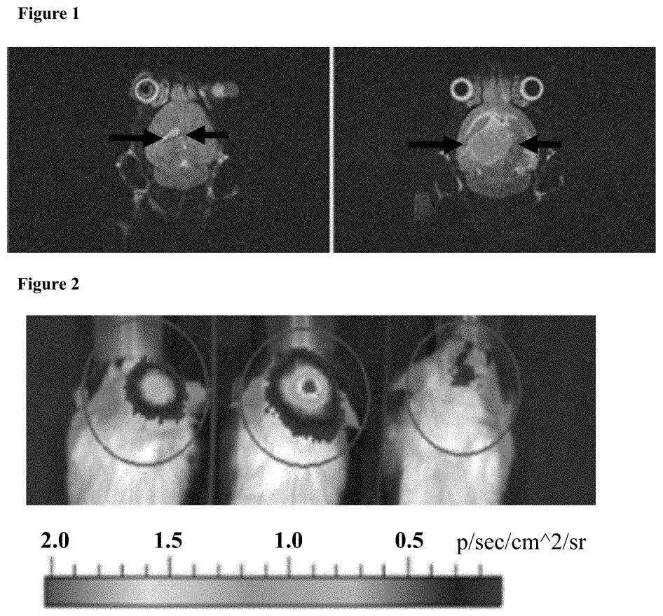

Eight days after transfer of U87 cells, tumors were visible using MRI ASPECT 1T imaging within the brains of the injected mice (FIG. 1, left panel). Exponential growth was visible on day 22 after injection, indicated the rapid and aggressive expansion of the glioblastoma cells (FIG. 1, right panel).

Curcumin and TMZ in targeted liposome carriers, were administered intraperitoneally to the mice 3 days after injection of the tumor cells and for at least 3 weeks. Both drugs were administered at 4 mg/kg per mouse, which is between 1/10- 1/30 of the dose that is commonly used for TMZ when treating glioblastoma.

### Example 3: Tumor Growth Was Inhibited, and the Development of the Cancer Was Delayed Following Treatment

TMZ-liposomes were administered daily to mice, as was free TMZ (no-liposomes) and saline as a control. The 4 mg/kg per mouse concentration of TMZ was well below levels reported to have an effect on glioblastoma growth and indeed there was no statistically significant difference in the growth of the tumor in mice that received free TMZ or saline (FIG. 2, left and middle mice). In contrast, daily administration of TMZ-liposomes resulted in a significant inhibition of tumor growth, with a near 10-fold reduction in the size of the tumor after 18 days (FIG. 2, right mouse).

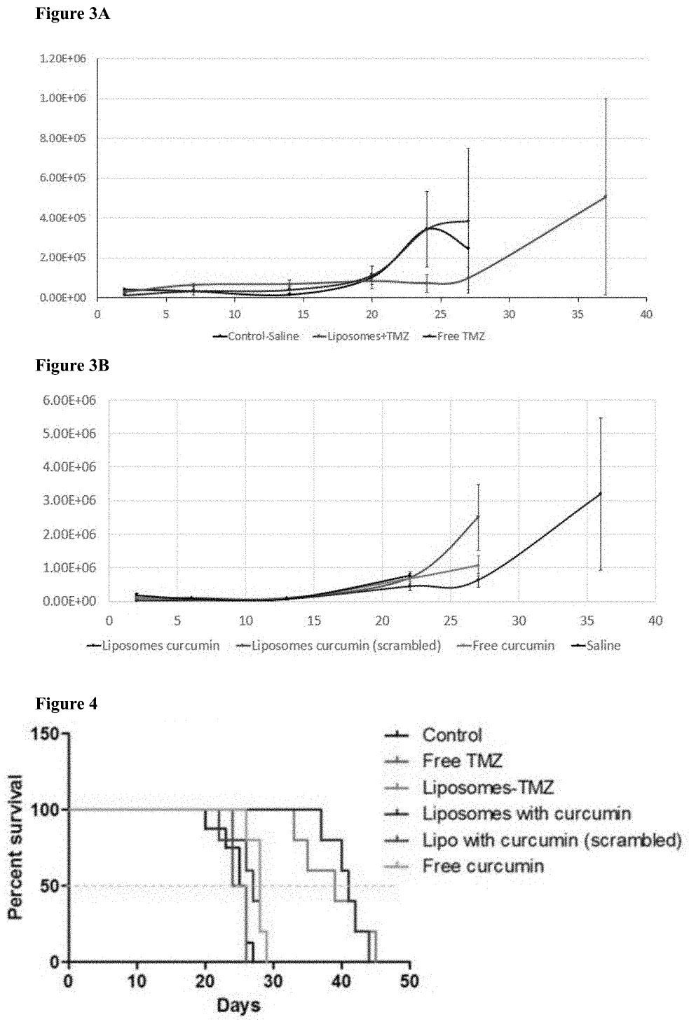

By using the luciferase imaging system, quantification of the relative tumor size was possible (FIG. 3). After transplant of the tumor cells there was a latency period in which little to no tumor growth occurred, although the cancer cells did emit fluorescence above background levels. Treatment with TMZ-liposomes (FIG. 3A) increased this latency period such that no increased growth was observed at the end of 3 weeks. In both the saline and free TMZ groups the latency period lasted only 2 weeks, with extensive growth beginning thereafter. TMZ-liposomes treatment extended latency until the middle of the 4^{th} week, and tumor growth comparable to that seen in the controls was not reached until the end of 5 weeks. Results with curcumin-liposomes (FIG. 3B) were similar, as comparable tumor growth was not seen until the end of the 5^{th} week. Additionally, dosing with curcumin-liposomes in which the targeting peptide had been scrambled, resulting in a loss of targeting to the BBB, did not significantly improve survival as compared to control.

### Example 4: TMZ-Liposomes and Curcumin-Liposomes Prolong Survival

Survival studies were also conducted in the U87 transferred mice (FIG. 4). All mice dosed with saline (black line) or free TMZ (red line) as control, died within 30 days of tumor cell transfer. Daily dosing with TMZ-liposomes (beige line) or curcumin-liposomes (blue line) extended survival by ˜60%. All treated mice lived past 30 days, with 50% surviving to about 40 days. When curcumin was administered without liposomes (free curcumin, pink line), or when curcumin was administered in liposomes with a scrambled targeting peptide (purple line) survival was not significantly extended, with all mice still dying before 30 days.

### Example 5: Stability and Selectivity of Current Liposomes is Enhanced by Increasing Phosphatidyl Phosphoric Acid Content to Above 10 Mol %

In order to maintain a negative zeta-potential experiments of drug or compound loading into/onto the liposomes were conducted. It was shown that the loading into the lipid or aqua compartments of the liposomes had a strong effect on the stability and the selectivity/efficacy of the different liposomes.

In these experiments it was shown that increasing the Phosphatidyl phosphoric acid (PA) to up to 20% w/w or mol % of the liposome had a dramatic positive impact in increasing both the stability and the selectivity/efficacy of the liposomes. Thus, increasing PA from 10% to 15% or 18% w/w or mol % had a positive effect on both the stability and the selectivity/efficacy of the liposomes.

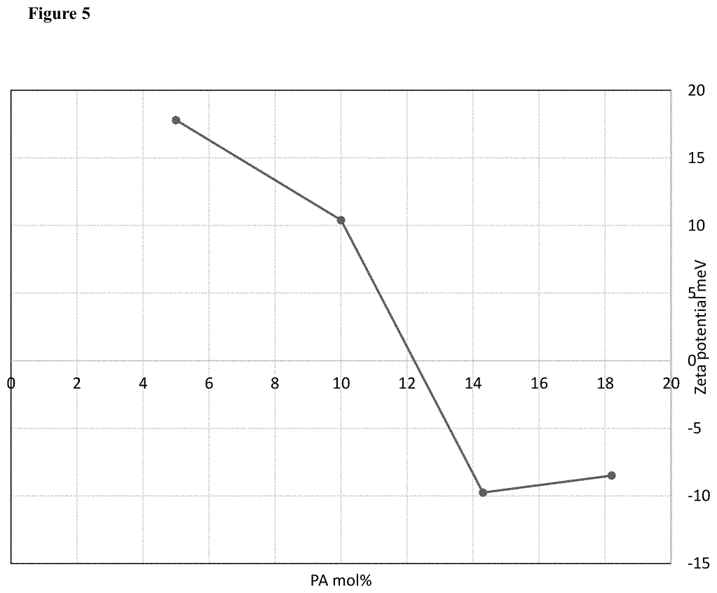

Specifically, an experiment conducted with liposomes having an increased content of PA and loaded with clorgyline (a specific inhibitor of Monoamine Oxidase A (MAO-A) revealed that the percentage of PA can be extended 15-18 mol % (see FIG. 5 and table 1).

Additionally, the stability of the liposomes loaded with temozolamin (TMZ) or Curcumin, both loaded in the lipid moiety of the liposomes was assessed.

Specifically, the stability of fresh and lyophilized liposome hydrated before the measurement kept at 4° C., was assessed.

Interestingly, no significant differences in size and/or charge of liposomes loaded with the drug kept lyophilized at 4° C. and hydrated before the measurement during the 3 months of measurements (liposomes loaded with curcumin seemed to have less variability than liposomes loaded with TMZ) was noticed.

Nonetheless, measuring hydrated liposomes, kept at 4° C. for to 3 months, revealed that although the zeta-potential of the hydrated liposomes was stable (did not changed significantly (up to 2%) during the 3 month), change in size of up to twice (largest diameter) was recorded after the first month of storage. No additional changes in size were observed between following the first month and up to 3 months.

These experiment, unexpectedly show the significant effect of elevated amount of PA on reaching/maintain negative zeta-potential and stability/selectivity.

TABLE 1

| Zeta | | | | |
|---|---|---|---|---|
| potential | Size | PA | Ege PC | Cholesterol |
| meV | nm | mol % | mol % | mol % |
| 17.8 | 195 | 5 | 55 | 40 |
| 10.4 | 134 | 10 | 50 | 40 |
| −9.76 | 156 | 14.3 | 47.6 | 38 |
| −8.5 | 187 | 18.2 | 45.5 | 36.3 |

1. A composition comprising a liposome, said liposome comprises 30 to 50% cholesterol, 5 to 20% phosphatidyl phosphoric acid and 40 to 60% phosphatidyl choline, by molarity.

2. The composition of claim 1, wherein said liposome further comprises a peptide anchored and conjugated to a succinate within said liposome bilayer, and wherein the peptide is exposed to the outer surface of the liposome.

3. The composition of claim 2, wherein said peptide comprises the amino acid sequence HRERMS (SEQ ID NO: 1), said succinate is 1,2-dioleoyl-sn-glycero-3-succinate and said peptide anchored and conjugated to 1,2-dioleoyl-sn-glycero-3-succinate comprises 0.1-2% of the liposome, by molarity.

4. A method for providing a composition capable of crossing a blood brain barrier (BBB), comprising the step of combining a drug or an imaging agent with the composition of claim 3, thereby providing a composition capable of crossing a BBB.

5. The composition claim 1, further comprising a drug or an imaging agent.

6. The composition of claim 5, wherein the liposome comprises 0.1 to 8% of said drug, by molarity.

7. The composition of claim 5, wherein the liposome comprises 0.1 to 10% of said imaging agent, by molarity.

8. The composition of claim 5, wherein said drug or said imaging agent is hydrophilic and encapsulated by said liposome.

9. The composition of claim 5, wherein said drug or said imaging agent is hydrophobic and embedded in the lipid layer of said liposome.

10. The composition of any one of claim 5, wherein said drug is a central nervous system (CNS) drug.

11.-12. (canceled)

13. The composition of claim 5, wherein said drug is selected from the group consisting of: curcumin, temozolomide (TMZ) or a combination thereof.

14. (canceled)

15. The composition of claim 1, wherein said 5 to 20% phosphatidyl phosphoric acid is 10 to 20% phosphatidyl phosphoric acid.

16. (canceled)

17. A method of treating or ameliorating a brain disease in a subject in need thereof comprising: administering a pharmaceutical composition comprising the composition of claim 5 and a pharmaceutically acceptable carrier or excipient to said subject, thereby treating said brain disease.

18.-23. (canceled)

24. The method of any one of claim 17, further comprising administering a cancer therapy selected from the group consisting of: radiation therapy, and chemotherapy.


SEQUENCE LISTING (4 sequences)

SEQ ID NO: 1 - protein, 6 aa, Homo sapiens
HRERMS

SEQ ID NO: 2 - protein, 5 aa, Homo sapiens
RERMS

SEQ ID NO: 3 - protein, 6 aa, Homo sapiens
ARERMS

SEQ ID NO: 4 - protein, 7 aa, Homo sapiens
AHRERMS